(19) United States
(12) Patent Application Publication
(10) Pub. No.: US 2022/0387987 A1
(43) Pub. Date: Dec. 8, 2022

(54) METHOD FOR PURIFYING NON-AQUEOUS SOLVENT AND METHOD FOR PRETREATING ION EXCHANGE RESIN FOR PURIFICATION OF NON-AQUEOUS SOLVENT

(71) Applicant: ORGANO CORPORATION, Tokyo (JP)

(72) Inventors: Yui SHIOYA, Tokyo (JP); Miwa ITO, Tokyo (JP); Noriko TAKADA, Tokyo (JP); Kaoru NUKUI, Tokyo (JP)

(73) Assignee: ORGANO CORPORATION, Tokyo (JP)

(21) Appl. No.: 17/771,673

(22) PCT Filed: Nov. 24, 2020
(86) PCT No.: PCT/JP2020/043559

§ 371 Date: Apr. 25, 2022

(30) Foreign Application Priority Data
Jan. 7, 2020 (JP) 2020-000851

(51) Int. Cl. B01J 39/05; B01J 47/04

(52) U.S. Cl. CPC B01J 39/05; B01J 47/04

(57) ABSTRACT

A method for purifying a non-aqueous solvent includes: pretreating by passing a non-aqueous solvent for dehydration treatment through a packed bed of an ion exchange resin that is not yet subjected to dehydration treatment, to remove water in the ion exchange resin; and purifying by passing a non-aqueous solvent to be purified through the packed bed of the ion exchange resin from which water is removed during the pretreating, to purify the non-aqueous solvent to be purified, in which the harmonic-mean particle size of the ion exchange resin is 0.20 to 0.50 mm. According to the present application, a method for purifying a non-aqueous solvent can be provided, the method includes pretreating by removing water in a water-containing ion exchange resin by passing therethrough a non-aqueous solvent for dehydration treatment, in which the amount of the non-aqueous solvent for dehydration treatment used during the pretreating is small.

### TECHNICAL FIELD

The present invention relates to a method for purifying a non-aqueous solvent for removing metallic impurities in the non-aqueous solvent to purify the non-aqueous solvent and a method for pretreating an ion exchange resin for purification of the non-aqueous solvent.

### BACKGROUND ART

Recently, in manufacturing semiconductors and lithium-ion secondary batteries, highly purified non-aqueous solvents have been used. As a method for purifying a non-aqueous solvent, a distillation method is known. However, this method has problems of requiring a high equipment cost and a great amount of energy, and also having difficulty in high purification.

In view of this, recently, a method for purifying a non-aqueous solvent has been performed by an ion exchange method using an ion exchange resin or an ion exchange filter. The ion exchange method has the characteristics of requiring a lower equipment cost, saving energy, and also enabling high purification.

In a highly purified non-aqueous solvent, water is considered to be an impurity, thus it is necessary to prevent water contained in an ion exchange resin from being eluted into the non-aqueous solvent. Thus, when an ion exchange resin is used, pretreatment to reduce the amount of water in the ion exchange resin in advance is needed.

For example, Patent Literature 1 discloses dehydration treatment of an ion exchange resin in which an anionic ion exchange resin is replaced with a water-miscible organic solvent and removed, and then this organic solvent is removed by deaeration. As another method therefor, a method of drying the ion exchange resin under reduced pressure is used.

### CITATION LIST

### Patent Literature

Patent Literature 1: Japanese Patent Publication H10-53594-A

### SUMMARY OF INVENTION

### Technical Problem

However, as a result of studies by the inventors of the present invention, it has been found that, in a method of reducing the water content in an ion exchange resin by passing therethrough a non-aqueous solvent for dehydration treatment and replacing water in the ion exchange resin with the non-aqueous solvent, even a several ten to several hundred times larger amount of the non-aqueous solvent than the amount of the ion exchange resin is required to reduce the water content in the ion exchange resin to such an extent that the non-aqueous solvent can be purified.

In a method of reducing the water content by drying an ion exchange resin under reduced pressure, the effect of reducing the water content in the ion exchange resin to a small amount of water for purification of the non-aqueous solvent to be performed is low.

Thus, it is an object of the present invention to provide a method for purifying a non-aqueous solvent and a method for pretreating an ion exchange resin, the method for purifying a non-aqueous solvent including a pretreatment step of removing water in a water-containing ion exchange resin by passing therethrough a non-aqueous solvent for dehydration treatment, in which the amount of the non-aqueous solvent used for dehydration treatment at the pretreatment step is small.

### Solution to Problem

Under this background, the inventors of the present invention have completed the present invention based on the findings that, for example, water in the ion exchange resin is less likely to be replaced with the non-aqueous solvent in a position closer to its center than near its surface at the pretreatment step, water near the center of the ion exchange resin is a factor in excessively increasing the amount of the non-aqueous solvent for dehydration treatment used at the pretreatment step, and accordingly water near the center of the ion exchange resin can be more easily replaced with the non-aqueous solvent by reducing the particle size of the ion exchange resin used for purification of the non-aqueous solvent to shorten the distance from the center to the surface.

Specifically, the present invention (1) provides a method for purifying a non-aqueous solvent, the method including:

a pretreatment step of passing a non-aqueous solvent for dehydration treatment through a packed bed of an ion exchange resin that is not yet subjected to dehydration treatment, to remove water in the ion exchange resin; and

a purification step of passing a non-aqueous solvent to be purified through the packed bed of the ion exchange resin from which water is removed at the pretreatment step, to purify the non-aqueous solvent to be purified, in which

a harmonic-mean particle size of the ion exchange resin is 0.20 to 0.50 mm.

The present invention (2) provides the method for purifying a non-aqueous solvent according to (1), in which a degree of cross-linking of the ion exchange resin is 4.0 to 8.0%.

The present invention (3) provides the method for purifying a non-aqueous solvent according to (1) or (2), in which the ion exchange resin is a gel-type ion exchange resin.

The present invention (4) provides the method for purifying a non-aqueous solvent according to any one of (1) to (3), in which the packed bed of the ion exchange resin is a mixed bed or a double bed of a strongly acidic cation exchange resin and one or more types selected from an ion exchanger other than the strongly acidic cation exchange resin, a synthetic adsorbent, and a porous adsorbent.

The present invention (5) provides a method for pretreating an ion exchange resin for purification of a non-aqueous solvent, the method including:

a pretreatment step of passing a non-aqueous solvent for dehydration treatment through a packed bed of an ion exchange resin that is not yet subjected to dehydration treatment, to remove water in the ion exchange resin, in which

a harmonic-mean particle size of the ion exchange resin is 0.20 to 0.50 mm.

The present invention (6) provides the method for pretreating an ion exchange resin for purification of a non-aqueous solvent according to (5), in which a degree of cross-linking of the ion exchange resin is 4.0 to 8.0%.

The present invention (7) provides the method for pretreating an ion exchange resin for purification of a non-aqueous solvent according to (5) or (6), in which the ion exchange resin is a gel-type ion exchange resin.

The present invention (8) provides the method for pretreating an ion exchange resin for purification of a non-aqueous solvent according to any one of (5) to (7), in which the packed bed of the ion exchange resin is a mixed bed or a double bed of a strongly acidic cation exchange resin and one or more types selected from an ion exchanger other than the strongly acidic cation exchange resin, a synthetic adsorbent, and a porous adsorbent.

### Advantageous Effects of Invention

According to the present invention, a method for purifying a non-aqueous solvent and a method for pretreating an ion exchange resin can be provided, the method for purifying a non-aqueous solvent including the pretreatment step of reducing the water content in the water-containing ion exchange resin by passing therethrough the non-aqueous solvent for dehydration treatment, in which the amount of the non-aqueous solvent used for dehydration treatment at the pretreatment step is small.

### DESCRIPTION OF EMBODIMENTS

A method for purifying a non-aqueous solvent according to the present invention includes:

a pretreatment step of passing a non-aqueous solvent for dehydration treatment through a packed bed of an ion exchange resin that is not yet subjected to dehydration treatment, to remove water in the ion exchange resin; and

a purification step of passing a non-aqueous solvent to be purified through the packed bed of the ion exchange resin from which water is removed at the pretreatment step, to purify the non-aqueous solvent to be purified, in which

the harmonic-mean particle size of the ion exchange resin is 0.20 to 0.50 mm.

The method for purifying a non-aqueous solvent according to the present invention includes the pretreatment step and the purification step. Specifically, in the method for purifying a non-aqueous solvent according to the present invention, the non-aqueous solvent is purified by passing the non-aqueous solvent for dehydration treatment in advance through the ion exchange resin that has not been subjected to dehydration treatment, thereby reducing the amount of water in the ion exchange resin, and then passing the non-aqueous solvent to be purified through the ion exchange resin in which the amount of water has been reduced.

In the method for purifying a non-aqueous solvent according to the present invention, the non-aqueous solvent for dehydration treatment to be used at the pretreatment step and the non-aqueous solvent to be purified at the purification step are preferably non-aqueous solvents of the same type, but may be of different types. If the non-aqueous solvent for dehydration treatment to be used at the pretreatment step and the non-aqueous solvent to be purified at the purification step are of different types, the purification step may be performed after passing the non-aqueous solvent to be purified through a granular resin having an ion-exchange group, which has been subjected to dehydration treatment, before the purification step and then replacing the non-aqueous solvent for dehydration treatment with the non-aqueous solvent to be purified. For example, to purify isopropyl alcohol, the isopropyl alcohol is used as the non-aqueous solvent for dehydration treatment. The non-aqueous solvent to be purified is not limited to a particular one, and examples thereof include: alcohols such as isopropyl alcohol, methanol, and ethanol; ketones such as cyclohexanone, methyl isobutyl ketone, acetone, and methyl ethyl ketone; alkene-based organic solvents such as 2,4-diphenyl-4-methyl-1-pentene and 2-phenyl-1-propene; ester-based organic solvents such as propylene glycol monomethyl ether acetate (PGMEA) and isopropyl acetate; aromatic organic solvents; N-methyl-pyrrolidone; and these organic solvents in combination.

The pretreatment step of the method for purifying a non-aqueous solvent according to the present invention is a step of passing the non-aqueous solvent for dehydration treatment through the packed bed of the ion exchange resin that is not yet subjected to dehydration treatment, to remove water from the ion exchange resin.

At the pretreatment step, as the non-aqueous solvent for dehydration treatment, a non-aqueous solvent the type of which is the same as or different from that of the non-aqueous solvent to be purified at the purification step is suitably selected. In terms of treatment capacity, a feed pipe is preferably arranged such that the non-aqueous solvent is passed as a downflow through the packed bed of the ion exchange resin packed in an ion exchange resin-packed container. When the non-aqueous solvent is passed as a downflow through the packed bed of the ion exchange resin, the non-aqueous solvent flowing through the packed bed of the ion exchange resin in the ion exchange resin-packed container is preferably adjusted to be pressurized such that bubbles are not generated in the ion exchange resin-packed container. In this case, as means (pressure adjusting means) for pressurizing the non-aqueous solvent, a back-pressure valve or a relief valve for pressurizing the ion exchange resin-packed container up to a predetermined pressure is preferably provided downstream of the ion exchange resin-packed container. By reducing the flowing fluid volume with the back-pressure valve or the relief valve to pressurize the ion exchange resin-packed container, bubbles can be prevented from being generated in the ion exchange resin-packed container.

The water content in the non-aqueous solvent for dehydration treatment only needs to be substantially the same as or be equal to or lower than a water content required for a non-aqueous solvent obtained by purification at the purification step. However, with a lower water content therein, the amount of the non-aqueous solvent needed for dehydration treatment can be reduced.

The content of each of metallic impurities in the non-aqueous solvent for dehydration treatment is suitably selected based on requirements for the non-aqueous solvent obtained by purification at the purification step. However, a metal content as low as possible is preferable from the viewpoint of reducing the consumption of functional groups of the ion exchange resin at the pretreatment step thereby increasing the useful life of the ion exchange resin.

In the method for purifying a non-aqueous solvent according to the present invention, the ion exchange resin that forms the packed bed of the ion exchange resin is a cation exchange resin or an anion exchange resin. The cation exchange resin may be a strongly acidic cation exchange resin, or may be a weakly acidic cation exchange resin. A cation exchange group introduced into the strongly acidic cation exchange resin is not limited to a particular one, and examples thereof include a sulfonate group. A cation exchange group introduced into the weakly acidic cation exchange resin is not limited to a particular one, and examples thereof include a carboxylic group. The cation exchange group of the cation exchange resin is preferably in the H⁺ form. The anion exchange resin may be a strongly basic anion exchange resin, or may be a weakly basic anion exchange resin. An anion exchange group introduced into the strongly basic anion exchange resin is not limited to a particular one, and examples thereof include a quaternary ammonium in the OH⁻ form. An anion exchange group introduced into the weakly basic anion exchange resin is not limited to a particular one, and examples thereof include a tertiary amino group, a secondary amino group, a primary amino group, and a polyamine group in the free base-form. The anion exchange group of the anion exchange resin is preferably in the free base-form.

Examples of a substrate resin of the ion exchange resin include styrene-divinylbenzene copolymers. The ion exchange resin is not limited to a particular one, but is preferably an organic polymer-based ion exchange resin having an organic polymer as a matrix, and examples of the organic polymer as a matrix include a styrenic resin and an acrylic resin.

The ion exchange resin may have any structure of a gel-type structure, a macroporous structure, and a porous structure.

The harmonic-mean particle size of the ion exchange resin is 0.20 to 0.50 mm, and is preferably 0.20 to 0.40 mm. By setting the harmonic-mean particle size of the ion exchange resin within this range, the amount of the non-aqueous solvent for dehydration treatment to be used at the pretreatment step can be reduced. However, if the harmonic-mean particle size of the ion exchange resin is less than this range, pressure difference in passage therethrough excessively increases, which makes it difficult for a non-aqueous solvent having a higher viscosity to pass therethrough. If the harmonic-mean particle size exceeds this range, it is difficult for the non-aqueous solvent to permeate to the center of the resin, and accordingly the amount of non-aqueous solvent for dehydration treatment to be used at the pretreatment step excessively increases. In the present invention, the harmonic-mean particle size of the ion exchange resin is a value measured with a laser diffraction particle size distribution analyzer.

The degree of cross-linking of the ion exchange resin, i.e., the degree of cross-linking of a resin that is a substrate of the ion exchange resin is preferably 4.0 to 8.0%, and more preferably 6.0 to 8.0%. By setting the degree of cross-linking of the ion exchange resin within this range, the effect of reducing the amount of the non-aqueous solvent for dehydration treatment to be used at the pretreatment step can be increased.

The exchange capacity of the ion exchange resin is preferably 0.6 to 3.0 eq/L-R, and more preferably 1.5 to 3.0 eq/L-R.

Examples of the type of the ion exchange resin include those, the harmonic-mean particle size of which is 0.20 to 0.50 mm, and more preferably 0.20 to 0.40 mm, selected from chromatography series manufactured by Organo Corporation, DOWEX manufactured by The Dow Chemical Company, DIAION UBK series manufactured by Mitsubishi Chemical Corporation, TOYOPEARL series manufactured by Tosoh Corporation, and chromatography resin MCK series manufactured by Samyang Corporation.

The packed bed of the ion exchange resin that is not yet subjected to dehydration treatment for the pretreatment step is a packed bed formed by packing the ion exchange resin that is not yet subjected to dehydration treatment in a layered manner into a treatment column, a treatment container, or the like. The diameter and the thickness of the packed bed are suitably selected based on the flow rate or the like of the non-aqueous solvent to be purified.

The packed bed of the ion exchange resin may be a single bed formed of a cation exchange resin or an anion exchange resin, may be a mixed bed of a cation exchange resin and an anion exchange resin, or may be a double bed formed of an upstream cation-exchange-resin layer and a downstream anion-exchange-resin layer. Examples of the packed bed of the ion exchange resin include a mixed bed of a strongly acidic cation exchange resin and a weakly basic anion exchange resin, a double bed formed of an upstream strongly acidic cation-exchange-resin layer and a downstream weakly basic anion-exchange-resin layer, a mixed bed of a strongly acidic cation exchange resin in the H⁺ form and a weakly basic anion exchange resin in the free base-form, and a double bed formed of an upstream strongly acidic cation-exchange-resin layer in the H⁺ form and a downstream weakly basic anion-exchange-resin layer in the free base-form.

The packed bed of the ion exchange resin may be a mixed bed or a double bed of a strongly acidic cation exchange resin and one or more types selected from an ion exchanger other than the strongly acidic cation exchange resin, a synthetic adsorbent, and a porous adsorbent. Examples of the ion exchanger other than the strongly acidic cation exchange resin include an organic porous cation exchanger and an organic porous anion exchanger. Examples of the synthetic adsorbent include resins matrices of which are classified as styrenic, acrylic, and phenolic ones. Examples of the porous adsorbent include activated carbon, zeolite, and silica gel.

Examples of the porous adsorbent include activated carbon powder (e.g., activated carbon powder described in Japanese Patent Publication 2016-132651-A) obtained by carbonizing and activating a resin such as a phenol resin. As a method of obtaining the activated carbon powder by carbonizing and activating a resin such as a phenol resin, for example, the following method is exemplified. Spherical resin raw powder such as spherical phenol resin powder is carbonized in a carbonization furnace, whereby spherical carbide powder is obtained. As carbonization conditions at this time, conditions that the raw powder is held at a temperature of 850° C. for 30 minutes in a nitrogen atmosphere, for example, can be exemplified. Subsequently, the obtained carbide powder is activated in an activation furnace. As activation conditions, conditions that water vapor is fed into the furnace and the carbide powder is held at a temperature of 850° C. for 5 to 24 hours, for example, can be exemplified. Activated carbon obtained by the activation can be classified so as to have a predetermined particle size as needed.

At the pretreatment step, a space velocity (SV) as a flow rate when the non-aqueous solvent for dehydration treatment is passed through the packed bed of the ion exchange resin is not limited to a particular one, and is suitably selected. However, it is preferably 1 to 100 L/L-resin/h, and more preferably 5 to 20 L/L-resin/h.

At the pretreatment step, the temperature when the non-aqueous solvent for dehydration treatment is passed through the packed bed of the ion exchange resin is not limited to a particular one, and is suitably selected. However, it is preferably 0 to 60° C., and more preferably 15 to 25° C.

At the pretreatment step, the water content in the non-aqueous solvent for dehydration treatment that has been passed through the packed bed of the ion exchange resin gradually decreases with the passed fluid volume of the non-aqueous solvent for dehydration treatment. Thus, passing the non-aqueous solvent for dehydration treatment through the packed bed of the ion exchange resin that is not yet subjected to dehydration treatment is continued until the water content in the non-aqueous solvent for dehydration treatment that has been passed through the packed bed of the ion exchange resin reaches a desired value. Herein, the passed fluid volume of the organic solvent for dehydration treatment at the pretreatment step is suitably selected based on a water content that the non-aqueous solvent to be purified is required to have.

The non-aqueous solvent for dehydration treatment used at the pretreatment step is discarded, or is reused as a non-aqueous solvent for dehydration treatment after water is removed therefrom.

The purification step of the method for purifying a non-aqueous solvent according to the present invention is a step of passing a non-aqueous solvent to be purified through a packed bed of an ion exchange resin that has been subjected to dehydration treatment, to purify the non-aqueous solvent to be purified.

The non-aqueous solvent to be purified contains, as metallic impurities, Li, Na, Mg, Al, K, Ca, Ti, Cr, Mn, Fe, Co, Ni, Cu, Zn, As, Sr, Ag, Cd, Ba, Pb, and the like. The content of each metallic impurity in the non-aqueous solvent to be purified is not limited to a particular one, but is about 100 mass ppb to 20 mass ppt.

The water content in the non-aqueous solvent to be purified is equal to or lower than a water content required for the non-aqueous solvent obtained by purification at the purification step.

At the purification step, the space velocity (SV) when the non-aqueous solvent to be purified is passed through the packed bed of the ion exchange resin that has been subjected to dehydration treatment is not limited to a particular one, and is suitably selected. However, it is preferably 1 to 100 L/L-resin/h, and more preferably 5 to 20 L/L-resin/h.

At the purification step, the temperature when the non-aqueous solvent to be purified is passed through the packed bed of the ion exchange resin that has been subjected to dehydration treatment is not limited to a particular one, and is suitably selected. However, it is preferably 0 to 60° C., and more preferably 15 to 25° C.

At the purification step, the non-aqueous solvent to be purified is passed through the packed bed of the ion exchange resin that has been subjected to dehydration treatment, whereby the non-aqueous solvent to be purified is purified.

The content of each metallic impurity in the non-aqueous solvent obtained by performing the method for purifying a non-aqueous solvent according to the present invention is suitably selected based on uses or required properties of the non-aqueous solvent, but is preferably 10 mass ppt or less.

In the ion exchange resin, water is present in a manner contained in an ion exchange group. When the ion exchange resin is subjected to dehydration treatment by bringing the non-aqueous solvent into contact with the ion exchange resin, water in the ion exchange resin is less likely to be removed in a position closer to the center of the ion exchange resin than near the surface thereof. In view of this, in the method for purifying a non-aqueous solvent according to the present invention, water near the center of the ion exchange resin can be easily removed by reducing the particle size of the ion exchange resin to be subjected to the pretreatment step to shorten the distance from the surface to the center. Thus, in the method for purifying a non-aqueous solvent according to the present invention, the amount of the non-aqueous solvent for dehydration treatment needed to be used at the pretreatment step can be reduced.

A method for pretreating an ion exchange resin for purification of a non-aqueous solvent according to the present invention includes a pretreatment step of passing a non-aqueous solvent for dehydration treatment through a packed bed of an ion exchange resin that is not yet subjected to dehydration treatment, to remove water in the ion exchange resin, in which the harmonic-mean particle size of the ion exchange resin is 0.20 to 0.50 mm, and preferably 0.20 to 0.40 mm.

The degree of cross-linking of the ion exchange resin is preferably 4.0 to 8.0%, and more preferably 6.0 to 8.0%. The ion exchange resin is preferably a gel-type ion exchange resin. The packed bed of the ion exchange resin is preferably a mixed bed or a double bed of a strongly acidic cation exchange resin and one or more types selected from an ion exchanger other than the strongly acidic cation exchange resin, a synthetic adsorbent, and a porous adsorbent.

A pretreatment step of the method for pretreating an ion exchange resin for purification of a non-aqueous solvent according to the present invention is the same as the pretreatment step of the method for purifying a non-aqueous solvent according to the present invention. Thus, the non-aqueous solvent for dehydration treatment, the ion exchange resin that is not yet subjected to dehydration treatment, the packed bed of the ion exchange resin that is not yet subjected to dehydration treatment, a synthetic adsorbent, a porous adsorbent, a method of passing the solvent, conditions for passing the solvent, and the like that are used at the pretreatment step of the method for pretreating an ion exchange resin for purification of a non-aqueous solvent according to the present invention are also the same as the non-aqueous solvent for dehydration treatment, the ion exchange resin that is not yet subjected to dehydration treatment, the packed bed of the ion exchange resin that is not yet subjected to dehydration treatment, the synthetic adsorbent, the porous adsorbent, the method of passing the solvent, the conditions for passing the solvent, and the like that are used at the pretreatment step of the method for purifying a non-aqueous solvent according to the present invention.

### EXAMPLES

The following describes the present invention in detail referring to Examples thereof. It should be noted that the present invention is not limited to Examples below.

### Example 1

In an acrylic column having an inner diameter of 16 mm and a height of 200 mm, 36 mL of wet ion exchange resin regenerated in the H⁺ form (strongly acidic cation exchange resin, gel-type, AMBERLITE (registered trademark) CR3220 manufactured by Organo Corporation, harmonic-mean particle size: 0.23 mm, degree of cross-linking: 8.0%) was packed.

Subsequently, isopropyl alcohol (IPA) for dehydration treatment having a water content indicated in Table 1 was passed at a space velocity of 5 L/L-resin/h into the column, a liquid flowing out of the column was collected for each of bed volumes (BV) as passed fluid volumes indicated in Table 1, and the water content therein was measured. The results are listed in Table 1.

### Example 2

The same process was performed as in Example 1 except that a wet ion exchange resin regenerated in the H⁺ form (strongly acidic cation exchange resin, gel-type, AMBERLITE (registered trademark) CR1310 manufactured by Organo Corporation, harmonic-mean particle size: 0.33 mm, degree of cross-linking: 6.0%) was used instead of the wet ion exchange resin regenerated in the H⁺ form (strongly acidic cation exchange resin, gel-type, AMBERLITE (registered trademark) CR3220 manufactured by Organo Corporation, harmonic-mean particle size: 0.23 mm, degree of cross-linking: 8.0%). The results are listed in Table 1.

### Example 3

The same process was performed as in Example 1 except that a wet ion exchange resin regenerated in the H⁺ form (strongly acidic cation exchange resin, gel-type, AMBERLITE (registered trademark) CR1320 manufactured by Organo Corporation, harmonic-mean particle size: 0.37 mm, degree of cross-linking: 6.0%) was used instead of the wet ion exchange resin regenerated in the H⁺ form (strongly acidic cation exchange resin, gel-type, AMBERLITE (registered trademark) CR3220 manufactured by Organo Corporation, harmonic-mean particle size: 0.23 mm, degree of cross-linking: 8.0%). The results are listed in Table 1.

### Comparative Example 1

The same process was performed as in Example 1 except that a wet ion exchange resin regenerated in the H⁺ form (strongly acidic cation exchange resin, gel-type, AMBERJET (registered trademark) 1020 manufactured by Organo Corporation, harmonic-mean particle size: 0.64 mm, degree of cross-linking: 8.0%) was used instead of the wet ion exchange resin regenerated in the H⁺ form (strongly acidic cation exchange resin, gel-type, AMBERLITE (registered trademark) CR3220 manufactured by Organo Corporation, harmonic-mean particle size: 0.23 mm, degree of cross-linking: 8.0%). The results are listed in Table 1.

### Comparative Example 2

The same process was performed as in Example 1 except that a wet ion exchange resin regenerated in the 1-1⁺ form (strongly acidic cation exchange resin, gel-type, AMBERJET (registered trademark) 1060 manufactured by Organo Corporation, harmonic-mean particle size: 0.66 mm, degree of cross-linking: 16.0%) was used instead of the wet ion exchange resin regenerated in the 1-1⁺ form (strongly acidic cation exchange resin, gel-type, AMBERLITE (registered trademark) CR3220 manufactured by Organo Corporation, harmonic-mean particle size: 0.23 mm, degree of cross-linking: 8.0%). The results are listed in Table 1.

### <Measurement of Harmonic-Mean Particle Size of Ion Exchange Resin>

Measurement was conducted with a laser diffraction particle size distribution analyzer Mastersizer 3000 (manufactured by Malvern Panalytical Ltd).

TABLE 1

| | | Water Content (ppm) | | | | |
|---|---|---|---|---|---|---|
| | | | | | Compara- | Compara- |
| | | Example | Example | Example | tive | tive |
| | | 1 | 2 | 3 | Example 1 | Example 2 |
| IPA for | | 24 | 20 | 26 | 26 | 23 |
| dehydration | | | | | | |
| treatment | | | | | | |
| Bed | 5 | 2,941 | 4,027 | 2,908 | 6,472 | 6,084 |
| Volume | 10 | 698 | 634 | 337 | 2,102 | 3,194 |
| (BV) | 15 | 108 | 85 | 69 | 841 | 1,924 |
| | 20 | 38 | 33 | 23 | 398 | 1,073 |
| | 25 | 30 | 29 | 29 | 206 | 763 |
| | 30 | 28 | 22 | 28 | 112 | 563 |
| | 35 | 23 | 23 | 22 | 68 | 437 |
| | 40 | — | — | — | 53 | 349 |
| | 45 | — | — | — | 38 | 290 |
| | 50 | — | — | — | 35 | 234 |
| | 55 | — | — | — | 30 | 188 |
| | 60 | — | — | — | 30 | 162 |
| | 65 | — | — | — | — | 140 |

In Examples 1 to 3, the amounts of IPA used until the water content in IPA discharged from the packed bed decreased to 30 ppm or lower were about 30 to 35 By. By contrast, in Comparative Example 1, the water content in IPA discharged from the packed bed was 68 ppm at the time when the amount of IPA used was 35 BV, and in Comparative Example 2, the water content in IPA discharged from the packed bed was 437 ppm at the time when the amount of IPA used was 35 By. From these results, it is found that, in the present invention, by reducing the particle size of the ion exchange resin within the specified range of the present invention, the amount of the non-aqueous solvent for dehydration treatment needed to be used at the pretreatment step can be reduced.

1. A method for purifying a non-aqueous solvent, the method comprising:
pretreating by passing a non-aqueous solvent for dehydration treatment through a packed bed of an ion exchange resin that is not yet subjected to dehydration treatment, to remove water in the ion exchange resin; and
purifying by passing a non-aqueous solvent to be purified through the packed bed of the ion exchange resin from which water is removed during the pretreating, to purify the non-aqueous solvent to be purified, wherein
a harmonic-mean particle size of the ion exchange resin is 0.20 to 0.50 mm.

2. The method for purifying the non-aqueous solvent according to claim 1, wherein a degree of cross-linking of the ion exchange resin is 4.0 to 8.0%.

3. The method for purifying the non-aqueous solvent according to claim 1, wherein the ion exchange resin is a gel-type ion exchange resin.

4. The method for purifying the non-aqueous solvent according to claim 1, wherein the packed bed of the ion exchange resin is a mixed bed or a double bed of a strongly acidic cation exchange resin and one or more types selected from an ion exchanger other than the strongly acidic cation exchange resin, a synthetic adsorbent, and a porous adsorbent.

5. A method for pretreating an ion exchange resin for purification of a non-aqueous solvent, the method comprising:
pretreating by passing a non-aqueous solvent for dehydration treatment through a packed bed of an ion exchange resin that is not yet subjected to dehydration treatment, to remove water in the ion exchange resin, wherein
a harmonic-mean particle size of the ion exchange resin is 0.20 to 0.50 mm.

6. The method for pretreating the ion exchange resin for purification of the non-aqueous solvent according to claim 5, wherein a degree of cross-linking of the ion exchange resin is 4.0 to 8.0%.

7. The method for pretreating the ion exchange resin for purification of the non-aqueous solvent according to claim 5, wherein the ion exchange resin is a gel-type ion exchange resin.

8. The method for pretreating the ion exchange resin for purification of the non-aqueous solvent according to claim 5, wherein the packed bed of the ion exchange resin is a mixed bed or a double bed of a strongly acidic cation exchange resin and one or more types selected from an ion exchanger other than the strongly acidic cation exchange resin, a synthetic adsorbent, and a porous adsorbent.
